(19) United States
(12) Patent Application Publication
(10) Pub. No.: US 2014/0094929 A1
(43) Pub. Date: Apr. 3, 2014

(54) INSERTION DEVICE FOR PLASTIC STENT

(71) Applicants: Kyong-Min Shin, Kyunggi-do (KR); TAEWOONG MEDICAL Co., LTD., Kyunggi-do (KR)

(72) Inventors: Kyong Min SHIN, Kyunggi-do (KR); YongHyun WON, Gyeonggi-do (KR)

(73) Assignees: TAEWOONG MEDICAL Co., LTD., Kyunggi-do (KR); Kyong-Min Shin, Kyunggi-do (KR)

(21) Appl. No.: 14/033,078

(22) Filed: Sep. 20, 2013

(30) Foreign Application Priority Data
Sep. 28, 2012 (KR) 10-2012-0108666

(51) Int. Cl. A61F 2/95; A61M 27/00; A61F 2/04

(52) U.S. Cl. CPC A61F 2/95; A61F 2/04; A61M 27/008
USPC 623/23.66; 623/23.7

(57) ABSTRACT

An insertion device for a plastic stent includes an operation unit which is operated to move a movable wire forward or backward, a tip portion connected to an end of the movable wire, a tube connected to the operation unit, and a plurality of expanding members which are connected between a plurality of tip end portions of the tip portion in an alternate manner. The expanding member is made of soft or flexible material so that it is contracted in a longitudinal direction and expanded in a radial direction when the tip portion is moved backward. Because the expanding members are expanded in diameter when the movable wire is moved backward by the operation of the operation unit, an external surface of the expanding member comes into contact with an internal surface of the plastic stent and is securely held by the internal surface of the plastic stent.

### CROSS-REFERENCE TO RELATED APPLICATIONS

The present application claims priority of Korean Patent Application No. 10-2012-0108666, filed on Sep. 28, 2012, which is incorporated herein by reference in its entirety.

### BACKGROUND OF THE INVENTION

1. Field of the Invention

The present invention relates to an insertion device for a stent and more particularly to an insertion device for a plastic stent which facilitates the adjustment of the operating position of the plastic stent by pulling the plastic stent in an internal duct of a human body after causing an expanding member of the insertion device to be contracted in a longitudinal direction and expanded in a radial direction.

2. Description of the Related Art

Generally, various kinds of plastic stents have been used to keep a blood vessel expanded or to broaden a strictured organic duct when the organic duct is stricuterd due to a lesion in various organs such as the biliary tract, throat airway, and ureter or due to temporal inflammation attributable to a scar generated in the process of treating a lesion using an endoscope in the body.

When placing the plastic stent into an organ of a human body, the plastic stent is first inserted into an insertion device and then inserted into the organ of the human body together with the insertion device. In this process, the plastic stent is repeatedly actuated to advance or retreat for accurate placement of the plastic stent to an intended position.

When using the operation technique of pushing the plastic stent for placement of the plastic stent, in order to readjust an operating position after the plastic stent is removed from the insertion device, the plastic stent needs to he inserted into the insertion device again and then inserted into the body. In this case, there is a problem that it is difficult to readjust the operating position.

In order to solve this problem, proposed is a technology of tying an insertion device and a plastic stent with a string first until adjustment of the operating position is finished and then untying the insertion device and the plastic stent so that the plastic stent will be separated from the insertion device after the adjustment of the operating position of the plastic stent is finished. However, this technology is disadvantageous in that the typing process is burdensome and positional adjustment of the plastic stent cannot be achieved if the string is accidently cut during the positional adjustment.

Accordingly, there has been a strong demand for an improved insertion device for a plastic stent by which the plastic stent can move along with the insertion device without being separated from the insertion device so that the operating position of the plastic stent can be precisely adjusted. To achieve this object, in the insertion device, an expanding member is expanded in a radial direction to come into tight contact with and to support an internal surface of the plastic stent when the insertion device is pulled back.

### CITATION LIST

Patent Literature 1: Korean Patent Unexamined. Publication No. 2002-0089342 titled “Stent Insertion Device”

### SUMMARY OF THE INVENTION

Accordingly, the present invention has been made keeping in mind the above problems occurring in the related art, and is intended to provide an insertion device for a plastic stent which can adjust the operating position of the plastic stent.

Further, the present invention serves to provide an insertion device for a plastic stent which has an expanding member which is contracted in the longitudinal direction in response to the operation of an operation unit and is simultaneously expanded in diameter so that an external surface of the expanding member comes into contact with and supports an internal surface of the plastic stent.

Yet further, the present invention serves to provide an insertion device for a plastic stent in which the insertion device and the plastic stent are not tied with a string but are independently installed so that the insertion device enables a simple adjustment of the position of the plastic stent.

Yet further, the present invention serves to provide an insertion device for a plastic stent which has an expanding member having a cylindrical shape or a coil spring shape in which an expanding member will be selectively adopted from among various types depending on the use.

In one aspect, the present invention provides an insertion device for a plastic stent which is used to insert the plastic stent into an internal duct of a human body such as a biliary tract or ureter, the insertion device including: an operation unit which includes a body and a movable portion and moves a movable wire forward and backward; a tip portion connected to an end of the movable wire; a tube connected to the operation unit; and an expanding member connected between the tube and the tip portion and made of a soft material, the expanding member being contracted in a longitudinal direction and expanded in a radial direction, when the tip portion is moved backward, wherein the movable wire is moved backward in response to an operation of the operation unit, the expanding member is expanded in diameter, which causes an external surface of the expanding member to come into contact with an internal surface of the plastic stent and the expanding member to be securely held by the internal surface of the plastic stent.

As described above, the present invention has the advantage that it is possible to adjust the operating position of the plastic stent.

Further, an expanding member is contracted in the longitudinal direction and is simultaneously expanded in diameter in response to an operation of an operation unit so that an external surface of the expanding member will come into contact with and support an internal surface of the plastic stent.

Furthermore, the insertion device and the plastic stent are not tied with a string but are independently installed. In this manner, the plastic stent will be simply adjusted in position.

Yet furthermore, expanding members having a cylindrical shape arid a coil spring shape are selectively used depending on use of the plastic stent.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a perspective view illustrating an insertion device of a plastic stent according to one embodiment of the present invention;

FIG. 2 is a partial perspective view partially illustrating the insertion device according to the present invention which includes a plurality of expanding members;

FIG. 3 is a perspective view illustrating a cylindrical expanding member according to the present invention;

FIG. 4 is a perspective view illustrating a cylindrical expanding member with wrinkles according to the present invention;

FIG. 5 is a perspective view illustrating a cylindrical expanding member with protrusions according to the present invention;

FIG. 6 is a perspective view illustrating an expanding member with a plurality of support strips according to the present invention;

FIG. 7 is a perspective view illustrating an expanding member with wrinkles on the support strips according to the present invention;

FIG. 8 is a perspective view illustrating an expanding member with wrinkles on the support strips according to the present invention;

FIG. 9 is a perspective view illustrating an insertion device for a plastic stent according to another embodiment of the present invention;

FIG. 10 is a perspective view illustrating a coil-shaped expanding member according to the present invention;

FIGS. 11A and 11B are perspective views illustrating a spiral expanding member according to the present invention;

FIG, 12A is a view illustrating a state in which the coil-shaped expanding member is enclosed by a synthetic resin tube and FIG. 128 is a view illustrating a state in which the coil-shaped expanding member is enclosed by a synthetic resin tube;

FIGS. 13A and 13B are operational cross-sectional views illustrating a state in which the cylindrical expanding member according to the present invention is expanded to support the plastic stent;

FIGS. 14A and 14B are operational cross-sectional views illustrating a state in which the cylindrical expanding member with wrinkles according to the present invention is expanded to support the plastic stent;

FIGS. 15A and 15B are operational cross-sectional views illustrating a state in which the cylindrical expanding member with protrusions according to the present invention is expanded to support the plastic stent;

FIGS. 16A and 16B are operational cross-sectional views illustrating a state in which the expanding member with a plurality of support strips according to the present invention is expanded to support the plastic stent;

FIGS. 17A and 17B are operational cross-sectional views illustrating a state in which the expanding member with wrinkles at the support strips according to the present invention is expanded to support the plastic stent;

FIGS. 18A and 18B are operational cross-sectional views illustrating a state in which the expanding member with protrusions at the support strips according to the present invention is expanded to support the plastic stent;

FIGS. 19A and 19B are operational cross-sectional views illustrating a state in which the coil-shaped expanding member is expanded to support the plastic stent; and

FIGS. 20A and 20B are operational cross-sectional views illustrating a state in which the spiral expanding member with wrinkles according to the present invention is expanded to support the plastic stent.

### DETAILED DESCRIPTION OF THE INVENTION

Hereinbelow, preferred embodiments of the invention will be described in detail with reference to the accompanying drawings. Unless otherwise defined, all terms used in this specification and claims, including technical or specific terms, should be contextually interpreted in light of the concept of the present invention and are not to be interpreted to have ideal or expressively formal meanings defined in a generally used dictionary based on the rule that inventors can define specific terms to most properly describe their inventions.

The structure described in the section ‘embodiments’ of this specification and drawings are only given in an exemplary way, and do not represent all of the technical concepts of the present invention. Accordingly, it can be understood that there may be various equivalents and modifications at the time when the present application is filed.

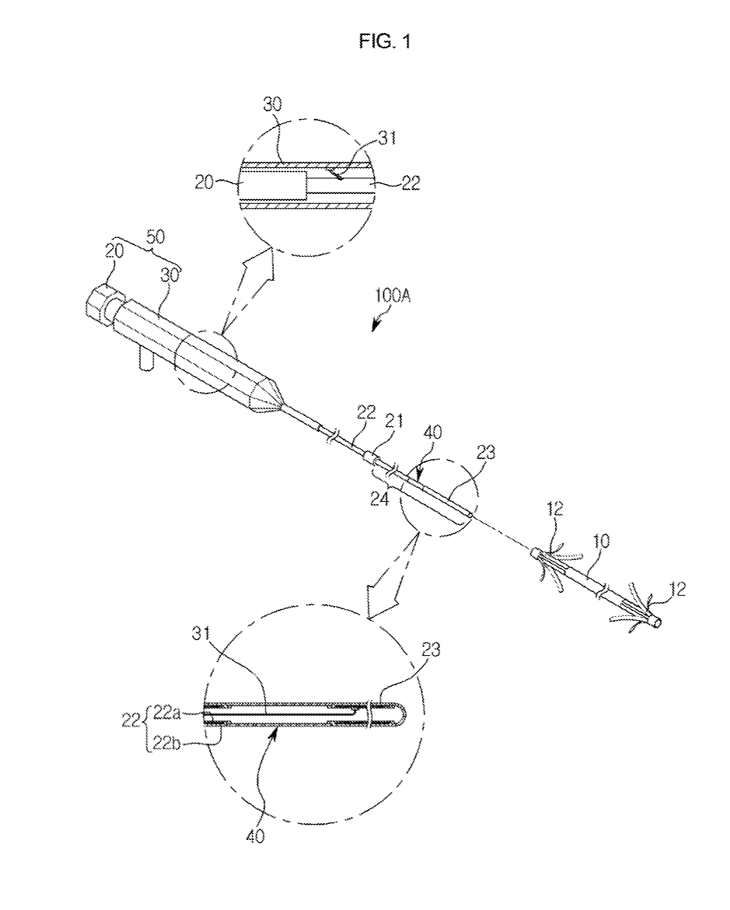

As illustrated in FIG. 1, an insertion device for a plastic stent is a device used to insert a plastic stent 10 into an internal duct such as a biliary tract or ureter. An insertion device 100A or 100B according to one embodiment of the present invention includes an operation unit 50. The operation unit 50 includes a body 20 and a movable portion 30 connected to each other. The insertion device 100A or 100B further includes a tube 22, an expanding member 40, and a tip portion 23 which are connected in series in this order. A rear end of the tube 22 is connected to a front end of the movable portion 30. The insertion device 100A or 100B yet further includes a movable wire 31 which extends in the tube 22 and is connected to the tip portion 23.

In the insertion devices 100A or 100B, a support protrusion 21 is provided on an external surface of the tube 22 in a position near the expanding member 40.

The body 20 and the movable portion 30 of the operation unit 50 are configured in such a manner that the movable wire 31 can be actuated to move forward and backward smoothly.

The tube 22 is connected to the front end of the body 20, and the support protrusion 21 is connected to the tube 22 in order to support the plastic stent 10 when the tube 22 is connected to the plastic stent 10 by being inserted into the plastic stent 10.

The tube 22 includes a coil-shaped metallic core 22 and a covering 22b which is made of nylon, Teflon® (trademark), or PEBAX® (trademark, polyEther Block Amides) and which encloses the coil-shaped metallic core 22.

The tube 22 can be freely deformed and restored to an original shape due to the metallic core 22a, and is less fractional when it is inserted into an internal duct of a human the body due to the covering 22b.

The tube 22 may be composed of only the metallic core 22a or only the covering 22b made of polymer. According to the present embodiment, the tube 22 is composed of the metallic core 22a and the covering 22b enclosing the metallic core 22a.

Preferably, the tip portion 23 has the same diameter as the tube 22. The tip portion 23 may be a part of the tube 22 or an additional tube or cylinder connected to the tube 22.

Preferably, the end of the tip portion 23 may be rounded.

The tip portion 23 may be connected to an end of the movable wire 31 which extends in the tube 22 and is connected to the movable portion 30. The movable wire 31 is actuated to advance or retreat in the tube 22.

For the purpose of increasing deformability and tension of the movable wire 31, the movable wire 31 may be formed by twisting plural strands of wire.

The movable portion 30 and the body 20 are connected to each other in such a manner that both of the movable portion 30 and the body 20 move forward and backward. The tip portion 23 connected to the movable wire 31 can move forward and backward along with the movement of the movable wire 31 within a movable range of the movable portion 30.

The expanding member 40 is connected between the tube 22 and the tip portion 23 and is structured to expand in a radial direction when the tip portion 23 is moved backward and the expanding member 40 is contracted in the longitudinal direction.

That is, the diameter D1 of the expanding member 40 before the expanding member 40 is expanded in the radial direction is smaller than an inner diameter d of the plastic stent 10, and the diameter D2 of the expanding member 40 after the expanding member 40 is contracted in the longitudinal direction and expanded in the radial direction is larger than the inner diameter d of the plastic stent 10. As a result, the expanding member 40 comes into tight contact with and comes to press the internal surface of the plastic stent 10.

The tube 22 is provided with an introduction hole through which a guide wire for guiding movement of the insertion device 100A or 100B is inserted into the tube 22. The guide wire then advances in the tube 22 and the expanding member 40, and passes through out a through-hole formed at the front end of the tip portion 23.

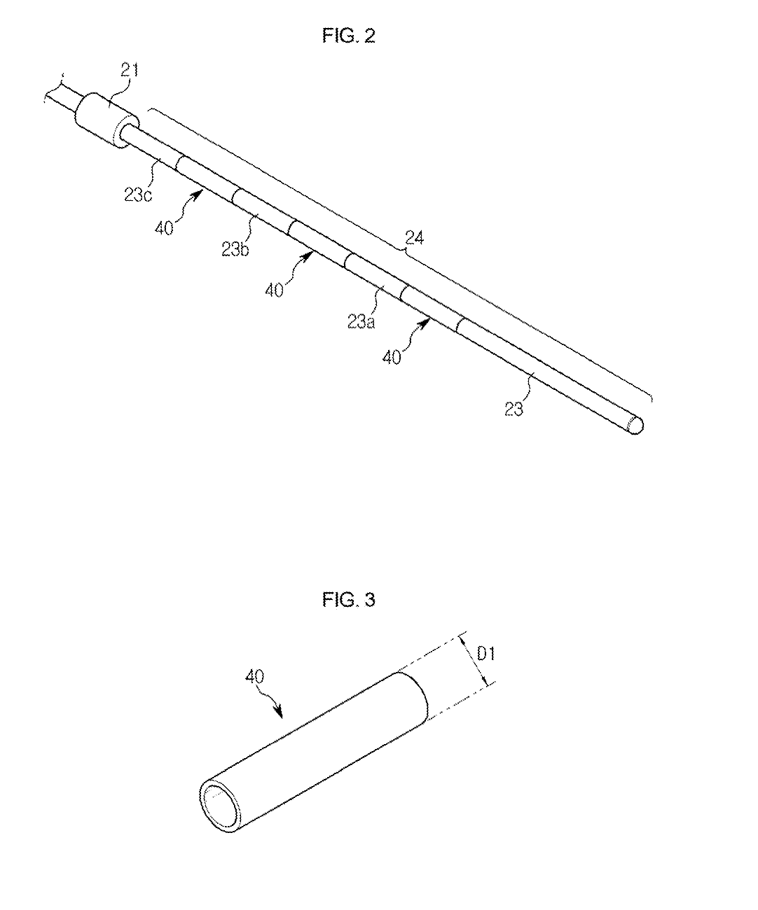

According to another embodiment, as illustrated in FIG. 2, a portion of the tip portion 23, to which the expanding member 40 is connected, is divided into a plurality of tip end portions 23a, 23b, 23c, . . .

The expanding member 40 may include a plurality of expanding members 40. Each of the expanding members 40 is connected between the tip portion 23 and a first tip end portion 23a, between adjacent two tip end portions 23b, 23c, . . . , and between a last tip end portion and the tube 22. A portion which is composed of the partial expanding members 40 and the tip end portions 23a, 23b, 23c, . . . of the tip portion 23 and which is to be inserted into the plastic stent 10 is called a loading portion 24.

When the movable wire 31 is moved backward in response to the operation of the body 20 and the movable portion 30, each of the expanding members 40 is contracted in the longitudinal direction and expanded in diameter. When the plastic stent 10 is increased in length, support force is distributed. The structure of the present invention enhances a support strength.

The expanding members 40 of the insertion device 100A are made of transparent or opaque synthetic resin with elasticity. According to the present embodiment, the expanding members 40 are made of soft synthetic resin, and features of various types of expanding members 40 are described below.

1. As shown in FIG. 3, the expanding member 40 has a cylindrical shape and can be freely expanded and contracted in the longitudinal direction. When the expanding member 40 is contracted in the longitudinal direction, the diameter of the expanding member 40 is increased.

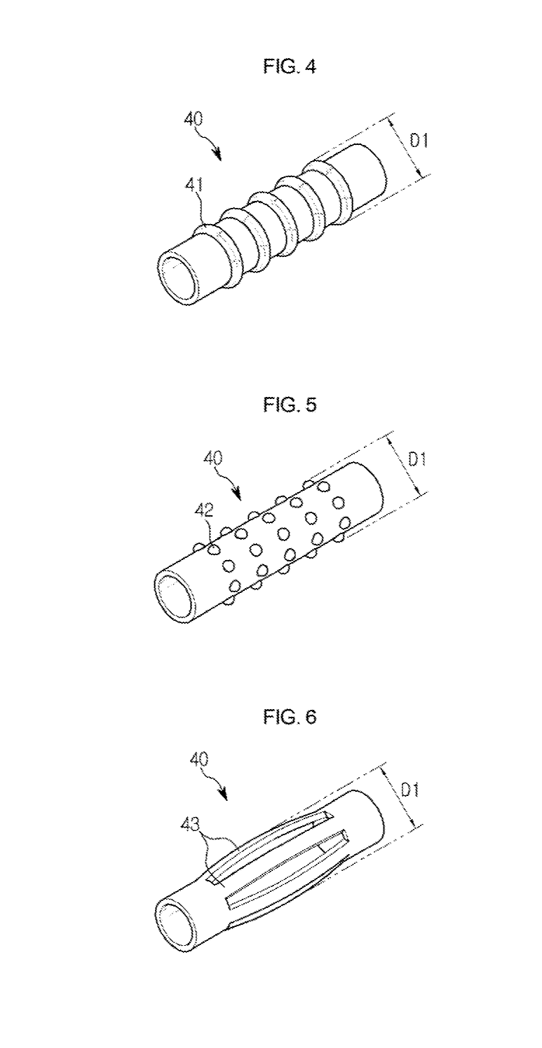

2. As shown in FIG. 4, the expanding member 40 has one or more wrinkles 41, each of which is continuous in the circumferential direction and is formed on an external surface. The wrinkles 41 increase frictional coefficient when the expanding member 40 comes into tight contact with an internal surface of the plastic stent 10.

3. As shown in FIG. 5, the expanding member 40 has one or more protrusions 42 on the external surface thereof. The protrusions 42 increase frictional coefficient when the expanding member 40 comes into tight contact with the internal surface of the plastic stent 10.

4. As shown in FIG. 6, a plurality of slits extending in the longitudinal direction are formed in the expanding sorter 40, with respective ends uncut, and two or more support strips 43 are provided between the slits in an alternate manner.

When the expanding member 40 is contracted in the longitudinal direction, the support strips 43 will be raised in a midway position.

Here, when the expanding member 40 is contracted in the longitudinal direction, the support strips 43 are raised, forming an outward convex curve, so that the support strips 43 will come into tight contact with the internal surface of the plastic stent 10 which has an inner diameter d.

5. As shown in FIG. 1, the expanding member 40 has one or more wrinkles 41 on external surfaces of the support strips 43. The wrinkles 41 increase the frictional coefficient when the expanding member 40 comes into tight contact with the internal surface of the plastic stent 10 which has the inner diameter d.

6. As shown in FIG. 8, the expanding member 40 has one or more protrusions 42 on the external surfaces of the support strips 43, The protrusions 42 increase frictional coefficient when the expanding member 40 comes into tight contact with the internal surface of the plastic stent 10 which has the inner diameter d.

That is, when the plastic stent 10 is moved backward after the plastic stent 10 was inserted into the internal duct by being pushed by the support protrusion 21, the expanding member 40 is contracted in the longitudinal direction and expanded in the radial direction by the backward movement of the movable portion 30. As a result, the expanding member 40 comes into tight contact with the internal surface of the plastic stent 10, so that the operating position of the plastic stent 10 can be adjusted.

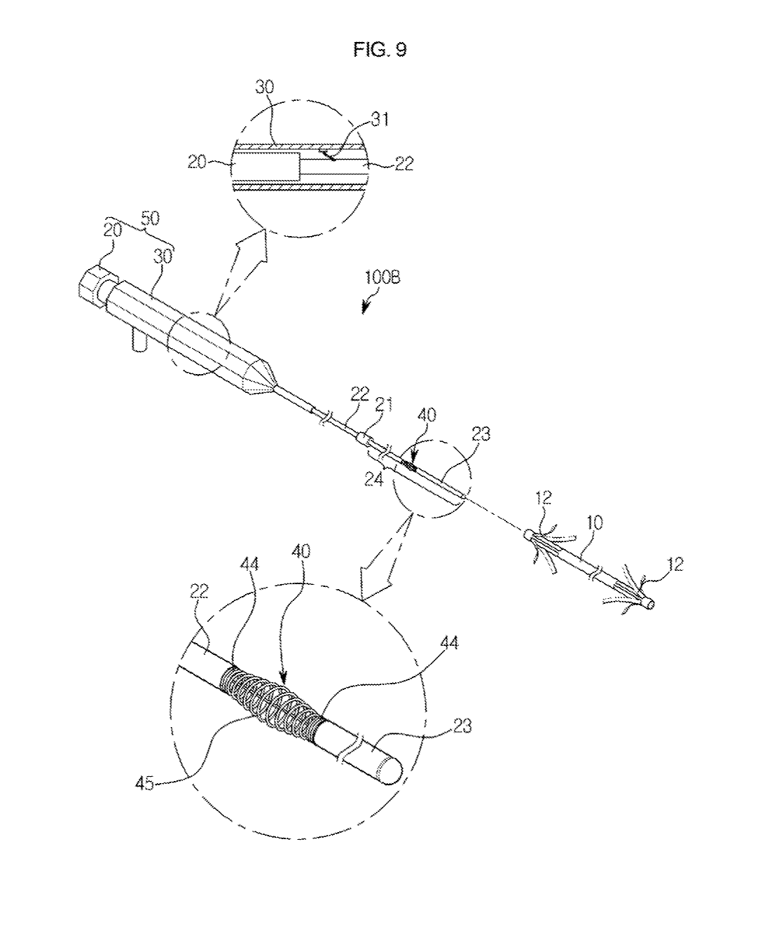

According to another embodiment, as shown in FIG. 9, the expanding member 40 of the insertion device 100B includes a coil spring which is made of elastic wire and which varies in diameter over its length. The coil spring has the largest diameter in a center position in the lengthwise direction. The features of the expanding member 40 according to the present embodiment are described below.

The wire used to form the expanding member 40 is an elastic metallic wire or an elastic synthetic resin wire.

1. As shown in FIGS. 9 and 10, the expanding member 40 is configured as a coil spring which is variable in diameter such that the diameter 45 (hereinafter, referred to as central diameter) in a center position in a longitudinal direction is larger than the diameter 44 (hereinafter, referred to as end diameter) at respective ends.

That is, when a user operates a body 20 and a movable portion 30 in order to pull a movable wire 31 (i.e., move the movable wire 31 backward), the expanding member 40 is contracted in the longitudinal direction and expanded in diameter in the center position. The diameter in the center position is referred to as “central diameter” 45 hereinafter.

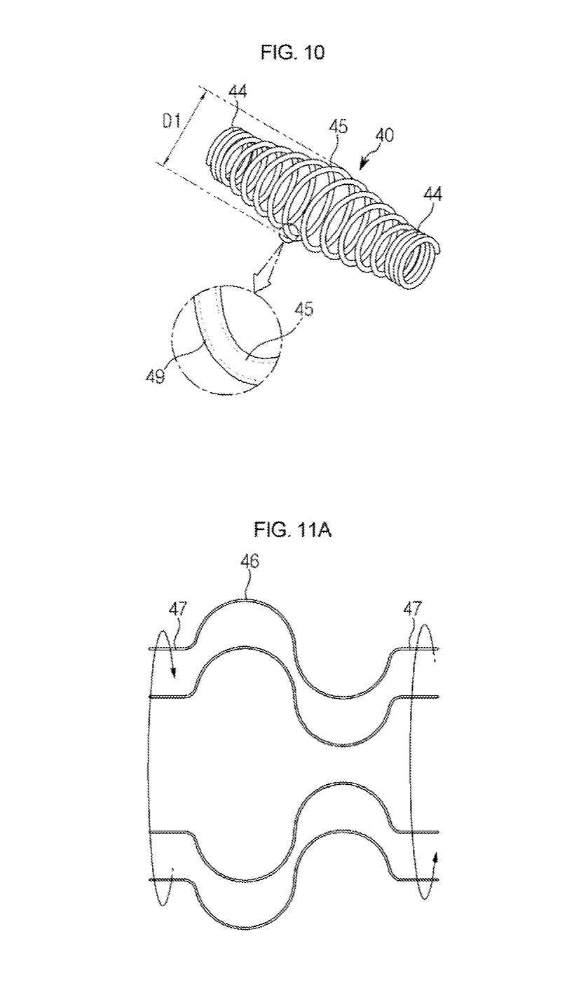

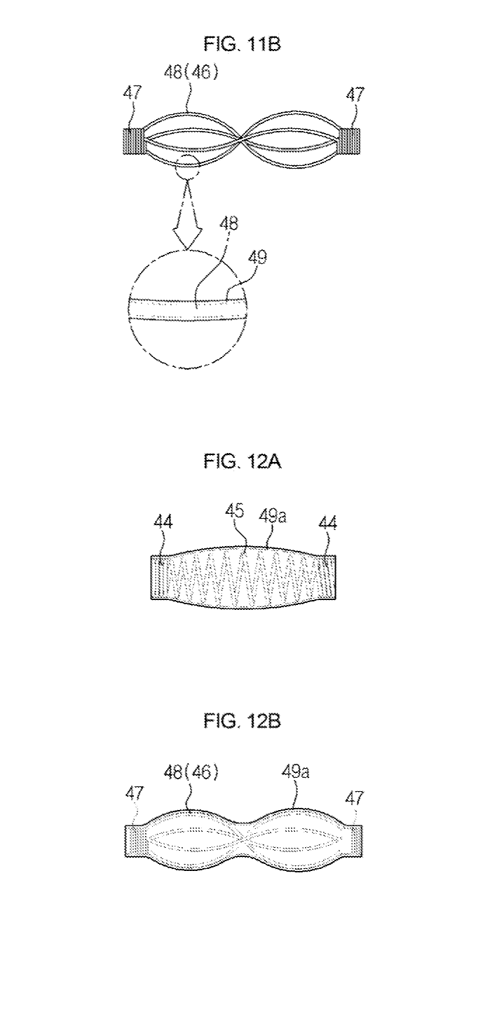

2. As shown in FIGS. 11A and 11B, the expanding member 40 may be an assembly of a plurality of elastic wires 47, each having one or more bent portions 46 and being bent in a spiral shape. The assembly of the elastic wires has a relatively small diameter at respective ends and a relatively large diameter in a center position. The portion having a large diameter is referred to as a radially expanded portion 48.

That is, when the body 20 and the movable portion 30 are operated and thus the movable wire 31 is pulled (i.e. moved backward), the expanding member 40 is contracted in the longitudinal direction and the radially expanded portion 48 is further expanded.

3. As shown in FIGS. 12A, the expanding member 40 is dipped in a silicone fluid and then cured so that a coating layer 49 will be formed on the surface of the expanding member 40. Alternatively, as shown in FIG. 12B, the expanding member 40 is enclosed with a soft silicone tube or a soft rubber tube 49a.

The coating layer 49 and the soft silicone tube or soft rubber tube 49a increases frictional coefficient and prevents the expanding member 40 from slipping along the surface of the plastic stent 10 when the expanding member 40 comes into contact the plastic stent 10.

That is, the expanding member 40 enters a first state in which the expanding member 40 is expanded in the longitudinal direction and an external surface of the radially expanded portion 48 in a position of the central diameter 45 is not in contact with the internal surface of the plastic stent 10 because the body 20 and the movable portion 30 is not operated after the plastic stent 10 was pushed by the support protrusion 21 of the insertion device and was inserted into the internal duct of a human body, and then enters a second state in which the expanding member 40 is contracted and compressed in the longitudinal direction and the external surface of the radially expanded portion 48 in the position of the central diameter 45 is in tight contact with the internal surface of the plastic stent 10 because the body 20 and the movable portion 30 are operated and the movable wire 31 is pulled back for backward movement of the plastic stent 10. In the second state, the external surface of the expanding member 40 is securely held by the internal surface of the plastic stent 10, so that the operating position can be adjusted.

Operation and advantages of the present invention, configured in the manner described above will be described below.

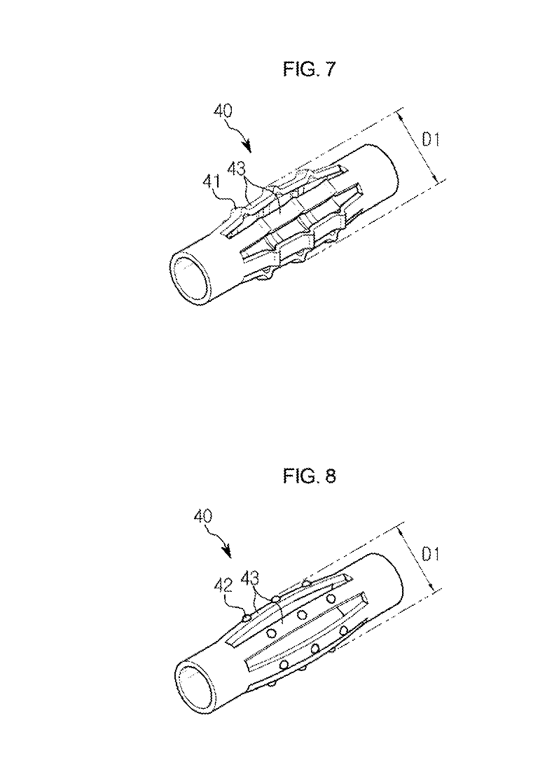

As shown in FIGS. 1 to 13B, in order to insert and place the plastic stent 10 in the internal duct such as a biliary tract using the insertion device 100A or 100B, the tip portion 23 and the tube 22 are inserted into the plastic stent 10 first.

When the tip portion 23 and the tube 22 are inserted, movement of a rear end of the plastic stent 10 is stopped by the support protrusion 21.

Then, the tube 22 is inserted into the biliary tract or ureter while being guided by a guide wire which was inserted in advance. At this time, as a device for securing the field of vision, an endoscope or an X-ray device may be used. The plastic stent 10 fixed to the insertion device 100A or 100B is inserted into the biliary tract or ureter, using this device.

Next, when the plastic stent 10 is inserted into the biliary tract or ureter in the operating position, the plastic stent 10 is inserted into an organ of a human body by being pushed by the support protrusion 21 combined with the tube 22.

In this way, if the plastic stent 10 is inserted into and placed in the biliary tract or ureter, the insertion device 100A or 100B is pulled back, and the plastic stent 10 is naturally separated from the tip portion 23 of the loading portion 24.

At this time, the plastic stent 10 does not move backward along with the insertion device 100A or 100B which is moved backward because a plurality of stoppers 12, which are bent such that an end portion of each stopper extends outward in the radial direction, are provided at each end portion of the plastic stent 10 and thus the support protrusion 12 is securely held by the internal surface of the biliary tract or ureter. In this state, the tube 22 of the insertion device 100A or 100B is pulled out of the plastic stent 10 and the insertion device 100A or 100B is fully removed from the human body, leaving the plastic stent 10 in an accurate operating position. In this way, the placement of the plastic stent 10 is completed.

When placing the plastic stent 10 in an internal duct of a human body, in order to precisely adjust the operating position of the plastic stent 10 by moving the insertion device 100A or 100B forward and backward, the body 20 of the operation unit 50 is moved forward and the movable portion 30 is simultaneously moved backward. Thus, the movable wire 31 and the tip portion 23 are pulled back together by the operation.

If the tip portion 23 is moved backward while the tube 22 stays in place, the expanding member 40 is contracted and compressed in the longitudinal direction and the tube 22 is folded to form ring-shaped wrinkles while the diameter of the expanding member 40 is increased from diameter D1 to diameter D2.

As the radially expanded portion having the diameter D2 comes into tight contact with and presses the internal surface of the plastic stent 10 having the inner diameter d, even when the insertion device 100A or 100B is moved backward, the plastic stent 10 is not separated from the tube 22 but is moved backward along with the backward movement of the insertion device 100A or 100B because the expanding member 40 is expanded and securely held by the internal surface of the plastic stent 10. In this way, the operating position of the plastic stent 10 is adjusted by simply moving the insertion device 100A or 100B backward or forward.

Operational features of the expanding member 40 used for the insertion device 100A vary depending on the shape.

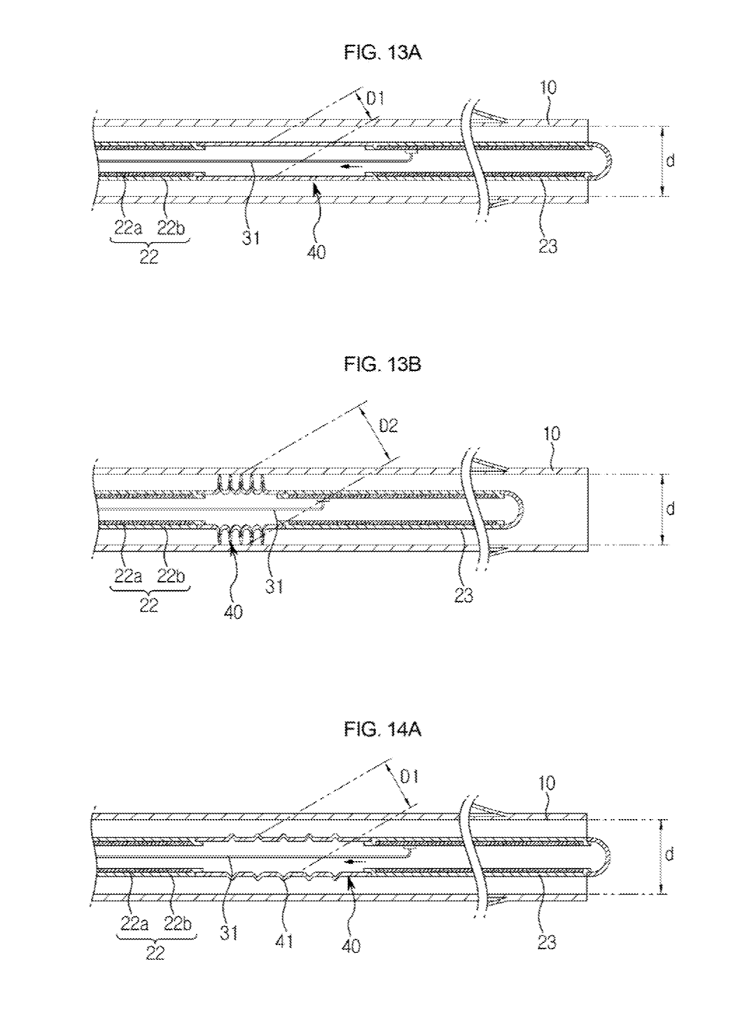

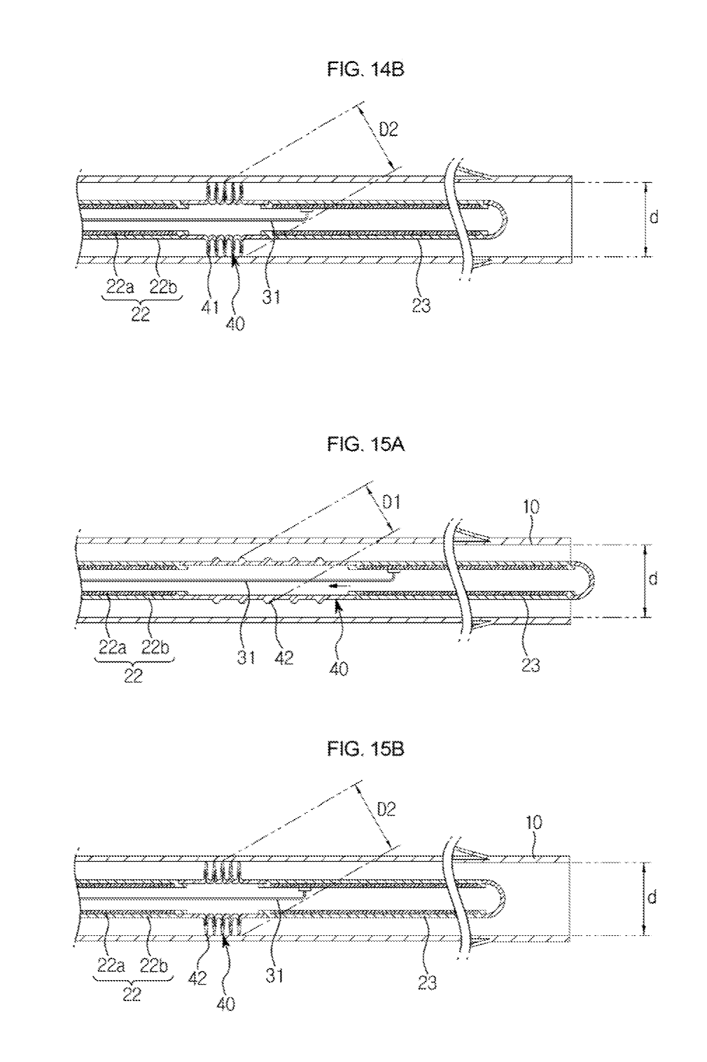

1. As shown in FIGS. 14A, 14B, 15A and 15B, when the expanding member 40 has wrinkles 41 or protrusions 42 on the external surface thereof, when the expanding member 40 comes into tight contact with the internal surface of the plastic stent 10 having the inner diameter d, the wrinkles 41 increase support force to support the plastic stent and the protrusions 42 increase frictional coefficient.

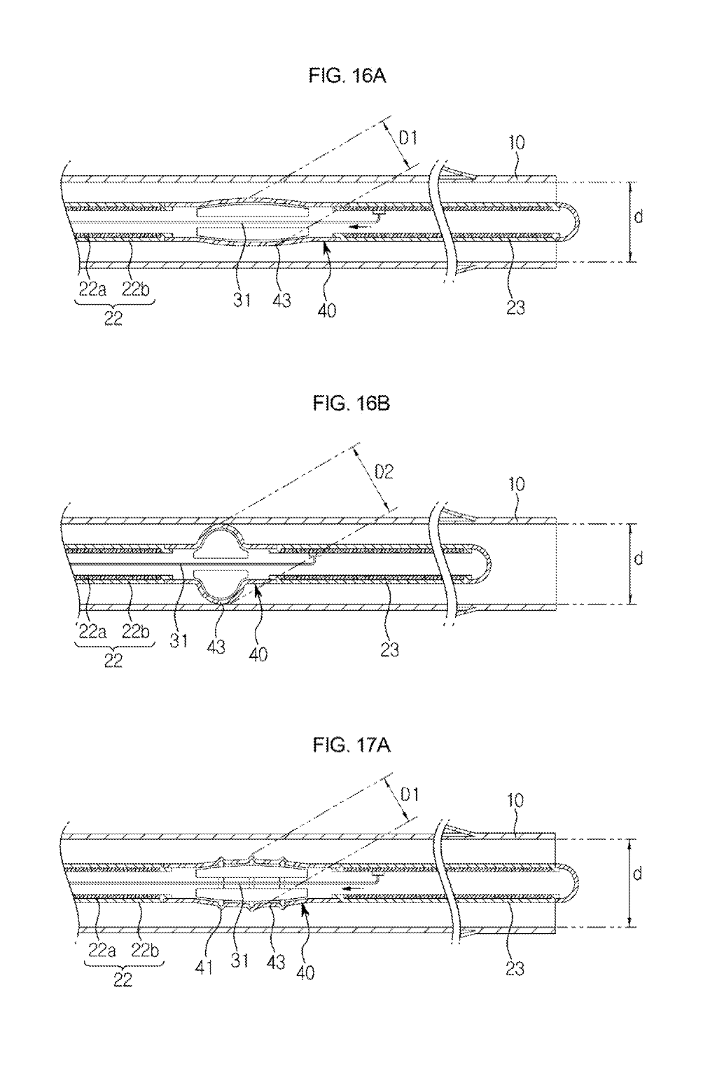

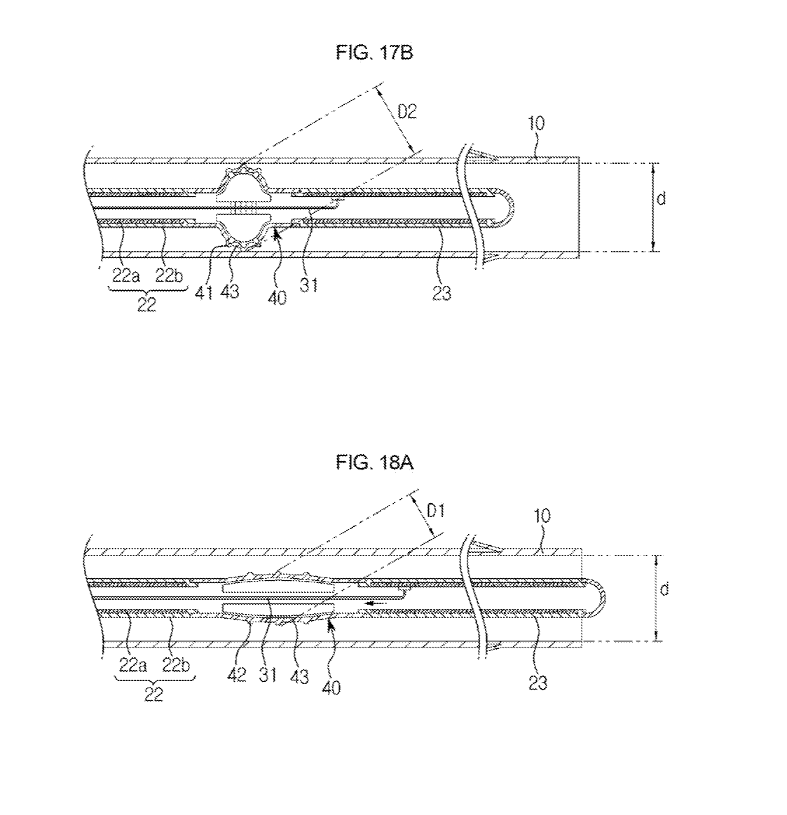

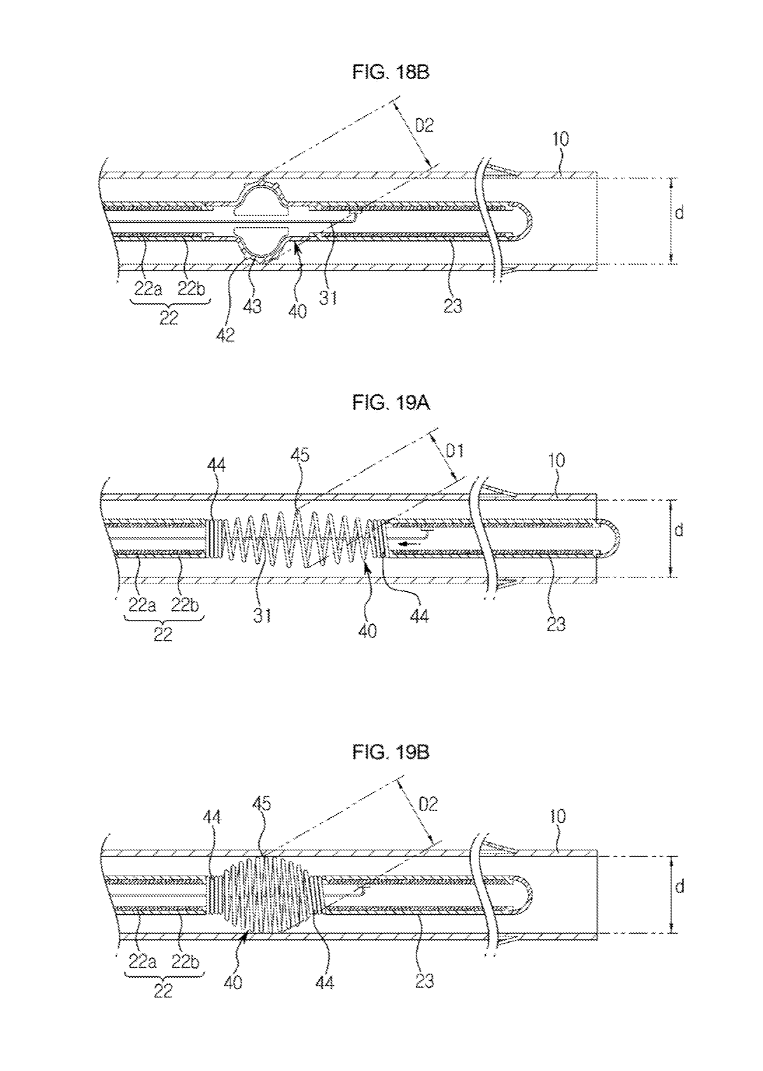

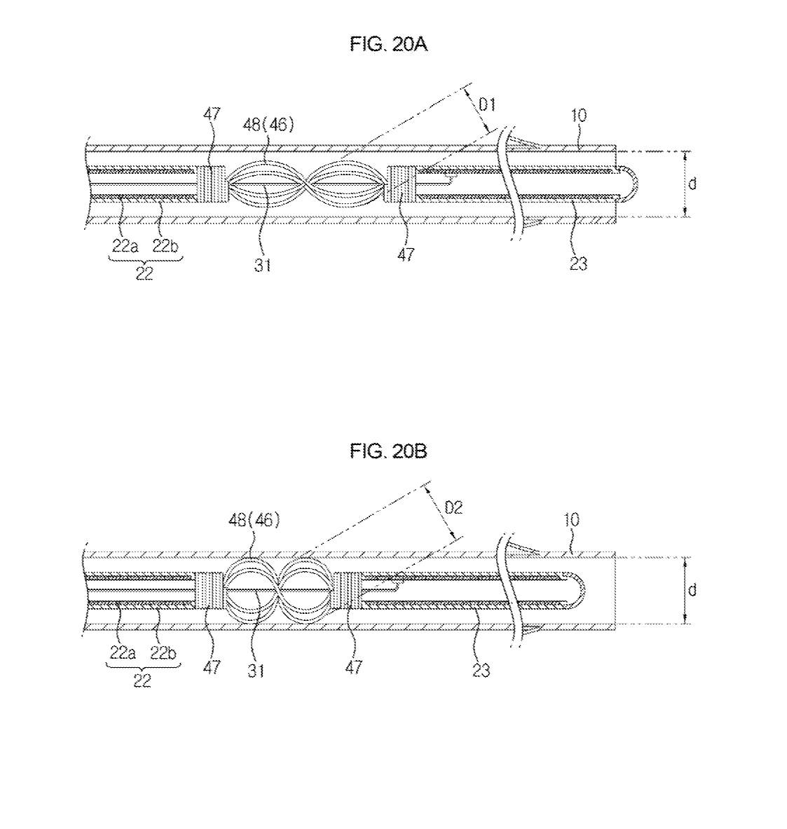

2. As shown in FIGS. 16A to 18B, when the expanding member 40 has support strips 43, the support strips 43 are raised in the form of a dome and thus the expanding member 40 comes into contact with and supports the internal surface of the plastic stent 10 having the inner diameter d. Fox the more, when the support strips 43 have wrinkles 41 or protrusions on the external surfaces thereof, frictional coefficient when the expanding member 40 comes into contact with the internal surface of the plastic stent 10 is increased.

According to a further embodiment of the present invention, as shown in FIGS, 19A, 19B, 20A and 20B, the expanding member 40 of the insertion device 10B has a coil spring shape. When the movable portion 30 is not operated, the expanding member 40 is in a longitudinally expanded state. In this state, a portion of the expanding member 40 in the position of the central diameter 45 or the radially expanded portion 46 is not in contact with the internal surface of the plastic stent 10.

If the movable portion 30 is moved backward in order to induce the backward movement of the plastic stent 10 in this state, the expanding member 40 which is in the longitudinally expanded state is contracted and compressed in the longitudinal direction and thus the portion of the expanding member 40 in the position of the central diameter 45 or the radially expanded portion 48 comes into contact with the internal surface of the plastic stent 10 and is securely held by the internal surface of the plastic stent 10. In this state, the operating position of the plastic stent 10 can be adjusted.

Because the expanding member 40 has a coil spring shape, the cohesive force is less concentrated when the expanding member 40 is contracted or expanded in the longitudinal direction. Accordingly, the insertion device according to the present invention is highly durable with respect to repetitive contraction and expanding operations.

Although a preferred embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. An insertion device for a plastic stent which is used to insert the plastic stent into an internal duct of a human body such as a biliary tract or ureter, the insertion device comprising;
an operation unit which includes a body and a movable portion and moves a movable wire forward and backward;
a tip portion connected to an end of the movable wire;
a tube connected to the operation unit; and
an expanding member connected between the tube and the tip portion and made of a soft material, the expanding member being contracted in a longitudinal direction when the tip portion is moved backward and being expanded in a radial direction;
wherein the movable wire is moved backward in response to an operation of the operation unit, the expanding member is expanded in diameter, which causes an external surface of the expanding member to come into contact with an internal surface of the plastic stent and the expanding member to be securely held by the internal surface of the plastic stent.

2. The insertion device for a plastic stent according to claim 1, wherein a portion of the tip portion to which the expanding member is connected, is divided into a plurality of end portions, and the tube is connected between the tip portion and each end portion, so that the expanding member is provided in plural in a loading portion to which the plastic stent is loaded.

3. The insertion device for a plastic stent according to claim 1, wherein the expanding member has a cylinder shape.

4. The insertion device for a plastic stent according to claim 3, wherein the expanding member has one or more wrinkles in order to increase friction between the expanding member and the plastic stent.

5. The insertion device for a plastic stent according to claim 3, wherein the expanding member has one or more protrusions on external surfaces thereof in order to increase friction between the expanding member and the plastic stent.

6. The insertion device for a plastic stent according to claim 3,
wherein a middle portion of the cylindrical expanding member is cut along a longitudinal direction in a plurality of positions so that a plurality of slits extending in the longitudinal direction and a plurality of support strips are arranged in an alternate manner in a circumferential direction, and
wherein the support strips are raised in an outward convex form when the expanding member is contracted in the longitudinal direction.

7. The insertion device for a plastic stent according to claim 6, wherein the support strips of the expanding member has one or more wrinkles on their external surfaces in order to increase frictional coefficient between the expanding member and the plastic stent.

8. The insertion device for a plastic stent according to claim 6, wherein the support strips of the expanding member has one or more protrusions on their strips in order to increase frictional coefficient between the expanding member and the plastic stent.

9. The insertion device for a plastic stent according to claim 1,
wherein the expanding member has a coil spring shape having variable diameters which are smallest in respective end positions and are gradually increased toward a center position, and
wherein the expanding member is contracted in the longitudinal direction and a diameter in the center position is further increased when the movable portion is pulled.

10. The insertion device for a plastic stent according to claim 1,
wherein the expanding member includes a plurality of elastic wires which are arranged in a spiral form and which have one or more bent portions,
wherein the expanding member has a relatively small diameter at respective end portions thereof and one or more radially expanded portions in a middle portion thereof, and
wherein the expanding member is contracted in the longitudinal direction and the radially expanded portion is expanded in a radial direction when the movable portion is pulled.

11. The Insertion device for a plastic stent according to claim 9,
wherein the expanding member is coated with a coating layer, which is formed by dipping the expanding member in a silicon fluid and curing the silicon fluid, or
wherein the expanding member is enclosed by a soft silicone tube or a soft rubber tube.
